(19) United States
(12) Patent Application Publication
(10) Pub. No.: US 2019/0049679 A1
(43) Pub. Date: Feb. 14, 2019

(54) OPTICAL CONNECTOR, LASER APPARATUS, ILLUMINATION APPARATUS, PROCESSING APPARATUS, THERAPEUTIC APPARATUS, AND OPTICAL COMMUNICATION APPARATUS

(71) Applicant: SHIMADZU CORPORATION, KYOTO (JP)

(72) Inventors: KAZUTOMO KADOKURA, KYOTO (JP); Mamoru HISAMITSU, KYOTO (JP); Kazuya INOUE, KYOTO (JP); Ryosuke NISHI, KYOTO (JP)

(73) Assignee: SHIMADZU CORPORATION, KYOTO (JP)

(21) Appl. No.: 16/076,481

(22) PCT Filed: Feb. 16, 2016
(86) PCT No.: PCT/JP2016/054445

§ 371 Date: Aug. 8, 2018

(51) Int. Cl. G02B 6/42; G02B 6/36; A61N 5/06; B23K 26/064

(52) U.S. Cl. CPC G02B 6/4292; G02B 6/3616; A61N 2005/067; B23K 26/064; A61N 2005/063; A61N 5/0616

(57) ABSTRACT

The optical connector includes an insert-opening 3b into which the optical fiber 4 is inserted, a connection-opening 3a that positions the tip of the optical fiber inserted into an insert-opening to a desired laser light connection position, and a guide 2a that guides the optical fiber inserted into the insert-opening 3f, where the insert-opening is in-place on the non-optical-axis of the laser light to be connected and a position-difference between the connection-opening and the insert-opening is larger than the predetermined value.

### CROSS REFERENCE TO RELATED APPLICATIONS

This application relates to, and claims priority from, Ser. No.:PCT/JP2016/054445 filed Feb. 16, 2016, the entire contents of which are incorporated herein by reference.

### FIGURE SELECTED FOR PUBLICATION

FIG. 1

### BACKGROUND OF THE INVENTION

### Field of the Invention

The present invention relates to an optical connector, a laser apparatus, an illumination apparatus, a processing apparatus, a therapeutic apparatus and an optical communication apparatus that are connected with an optical fiber and particularly, relates to the optical connector that guides laser light into the optical fiber.

### Description of the Related Art

A laser apparatus connecting with a laser light source and an optical fiber is widely used. In addition, the laser apparatus of which the optical fiber is attachable to the laser light source and detachable therefrom is also used. With regard to such a laser apparatus, the optical connector having a light-shutter mechanism to ensure safety in use, particularly for an eye, is proposed.

FIG. 7 is a cross-section view illustrating a conventional optical connector (Patent Document 1). Referring to FIG. 7, the optical connector comprises a lid-housing and the lid-housing houses optical shutter members 102, which opens upward and downward, and springs 103 adds pressure to the lid-housing and the optical shutter member 102.

In such aspects, once the optical fiber connector 100 travels toward left and contacts the optical shutter members 102, the optical shutter members 102 opens upward and downward and subsequently, the light travels toward right.

On the other hand, once the optical fiber connector 100 travels toward right and is taken out from the optical shutter 102, the optical shutter members 102 closes and therefore, traveling of the light toward right is blocked.

### RELATED PRIOR ART DOCUMENTS

### Patent Document

### Patent Document 1

### JP H8-320424 A1

### ASPECTS AND SUMMARY OF THE INVENTION

### Objects to be Solved

However, it is problematic that the mechanical shutter mechanism disclosed in Patent Document 1 has the following issues to be solved. Specifically, such as the shutter and the power (moving) units such as a spring to open and close the shutter are added. so that the structure thereof is more complicated. In addition, the optical connector is miniature, so that assembly of the optical connector is too hard. In addition, when the power unit such as springs are troubled, the light blocking function is incomplete.

The present invention relates to an optical connector, a laser apparatus, an illumination apparatus, a processing apparatus, a therapeutic apparatus and an optical communication apparatus that facilitate attaching-and-detaching to an optical fiber, controlling the output of laser light with no power unit for opening and closing and a simple structure and improving the safety therefor.

### Means for Solving the Problem

For solving the above problem, an optical connector, according to the present invention, comprises: a insert-opening into which an optical fiber is inserted; and a connection-opening that achieves positioning a tip of the optical fiber inserted into the insert-opening to a desired laser light connection position, wherein the insert-opening is in-place on the non-optical-axis of the laser light to be connected and the position-difference between the insert-opening and the connection-opening and relative to the optical-axis of the laser light is larger than a predetermined value.

### Effect of the Invention

According to the optical connector of the present invention, when the laser light emits, the optical fiber is inserted into the insert-opening and the tip of the optical fiber reaches to the connection-opening. Then the laser light propagates (travels) inside the optical fiber and the laser output-light is irradiated.

On the other hand, in the case of blocking the laser light, the laser light that scatters or travels in the straight line inside the connection-opening and passes therethrough contacts the proximity of the insert-opening, which is different from the laser optical-axis, and scatters and results in diminishing or blocking of the laser light when the optical fiber is taken out.

Accordingly, the optical connector, the laser apparatus, the illumination apparatus, the processing apparatus, the therapeutic apparatus and an optical communication apparatus that facilitate attaching to an optical fiber and detaching therefrom, controlling the output of laser light with no power unit for opening and closing and a simple structure and improving the safety therefor are provided.

The above and other aspects, features and advantages of the present invention will become apparent from the following description read in conjunction with the accompanying drawings, in which like reference numerals designate the same elements.

### BRIEF DESCRIPTION OF THE DRAWINGS

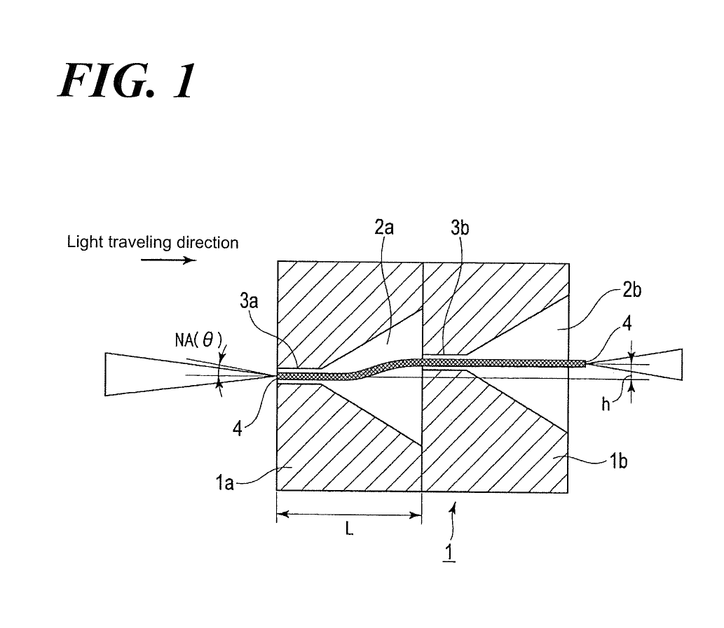

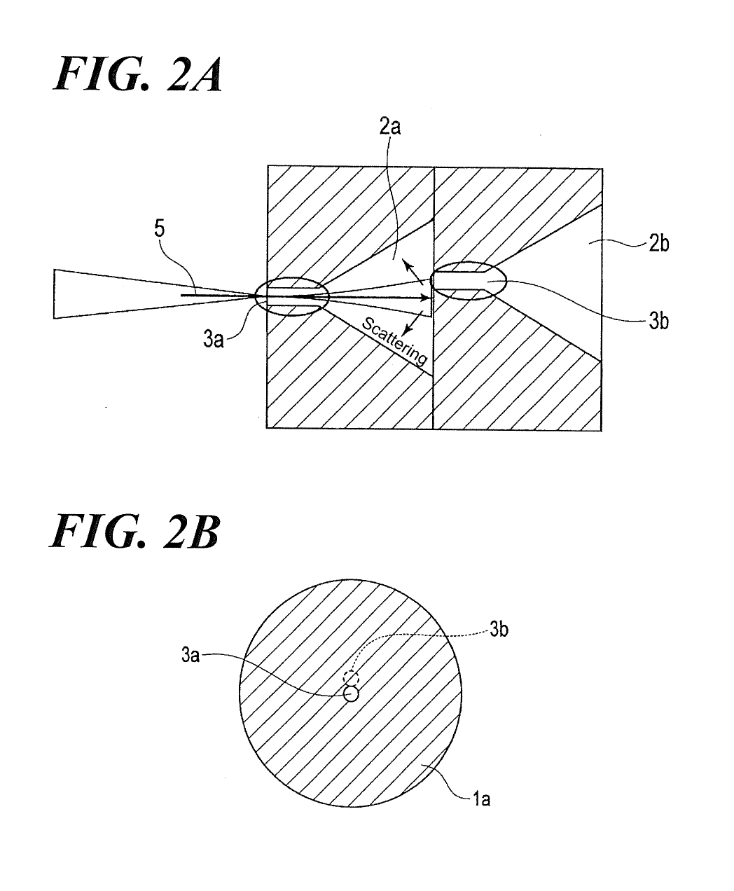

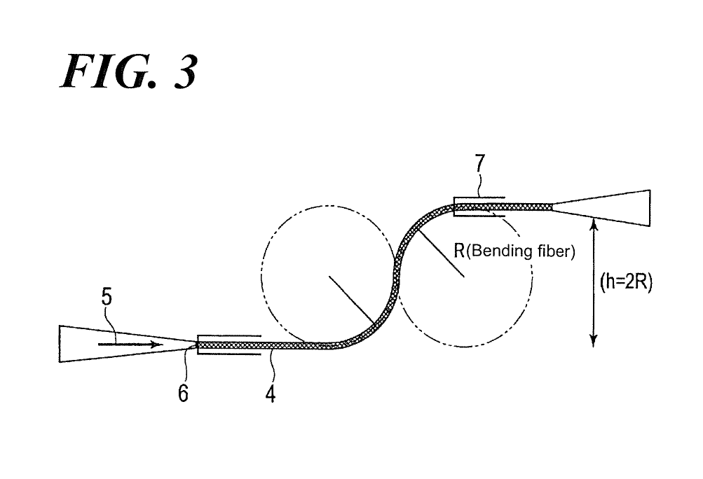

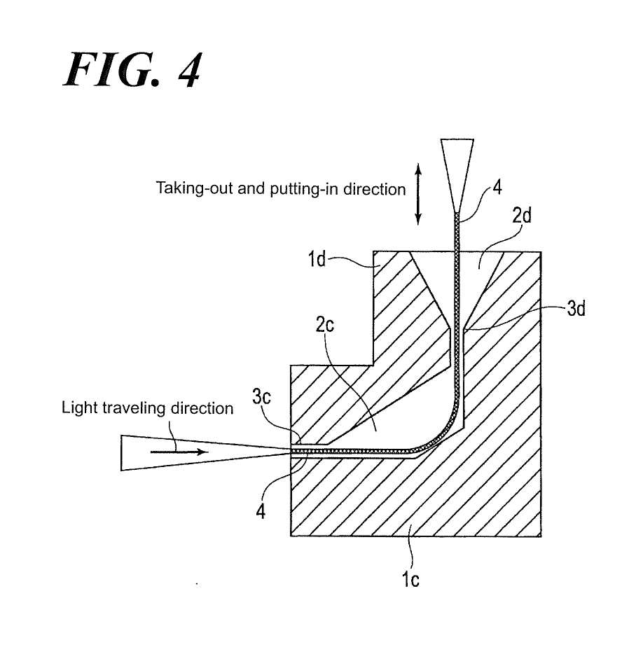

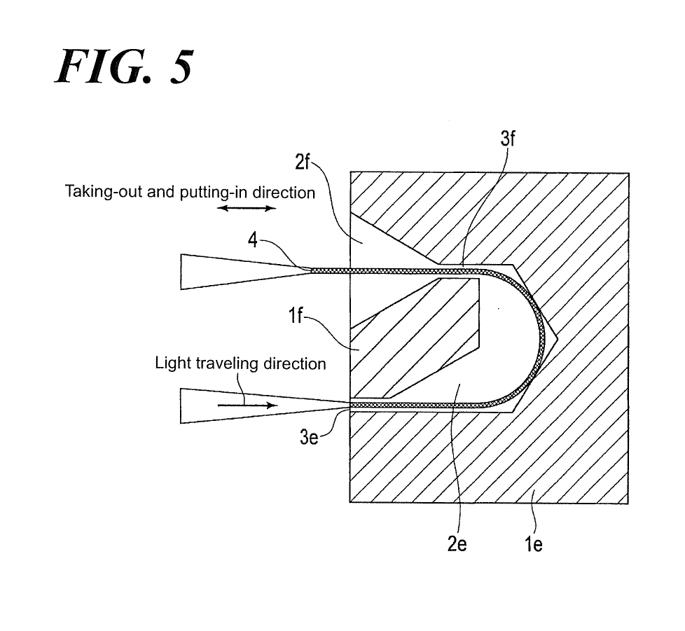

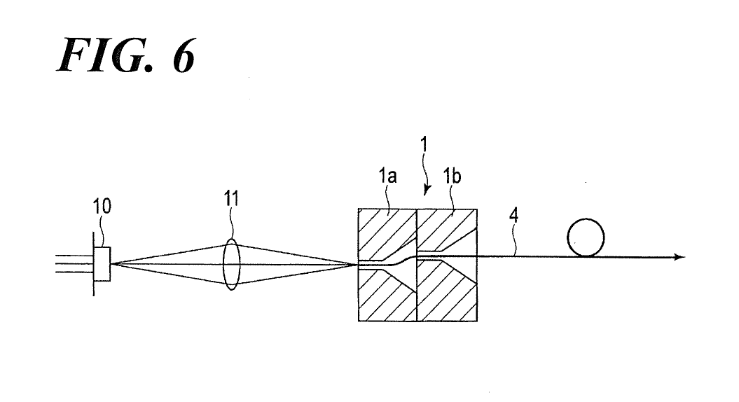

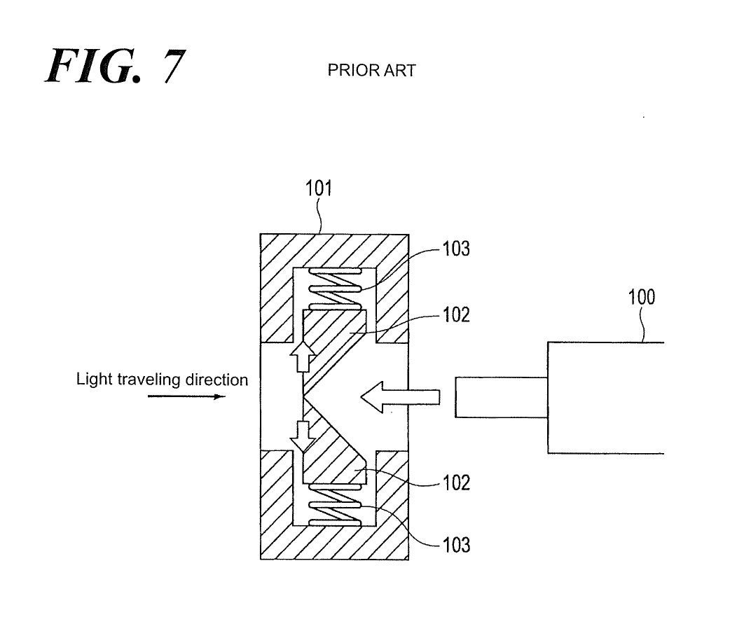

FIG. 1 is a cross-section view of the optical connector according to the aspect of the Embodiment 1 when the laser light emits.

FIGS. 2A and 2B are cross-section views of the optical connector according to the aspect of the Embodiment 1 when the laser light is blocked.

FIG. 3 is a view illustrating an application example of fiber-wiring inside the optical connector according to the aspect of the Embodiment 1.

FIG. 4 is a view illustrating fiber-wiring inside the optical connector according to the aspect of the Embodiment 2.

FIG. 5 is a view illustrating fiber-wiring inside the optical connector according to the aspect of the Embodiment 3.

FIG. 6 is a diagram illustrating a structure of the laser device using the optical connector according to the aspect of the Embodiment 1.

FIG. 7 is a cross-section view illustrating a conventional optical connector.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

Hereinafter, referring to FIGs., the inventors set forth further detail of an optical connector according to the aspect of the Embodiment of the present invention.

### Embodiment 1

FIG. 1 is the cross-section view of the optical connector according to the aspect of the Embodiment 1 when the laser light emits. Referring to FIG. 1, the laser light travels from left to right.

The optical connector 1 according to the aspect of the Embodiment 1 comprises an optical adapter 1a and an optical adapter 1b. The optical adapter 1a and the optical adapter 1b are made of easily processable metal material such as copper, aluminum and so forth.

The optical adapter 1b comprises an insert-opening 3b into which the optical fiber 4 is inserted and a first guide 2b having a conical shape to guide the optical fiber 4 to the insert-opening 3b. An opening-angle of the conical-shape first guide 2b is processed to be e.g., 60-degrees and the hole (opening) diameter of the insert-opening 3b is slightly larger than the diameter of the optical fiber.

The optical adapter 1a comprises a connection-opening 3a that achieves positioning the tip of the optical fiber 4 inserted into the insert-opening 3b to the desired laser light connection position and a second guide 2b having a conical shape to guide the optical fiber 4, which is inserted into the insert-opening 3b, to the connection-opening 3a. An opening-angle of the conical-shape second guide 2b is processed to be e.g., 60-degrees and the hole (opening) diameter of the connection-opening 3a is slightly larger than the diameter of the optical fiber. The cross-section of the connection-opening 3a and the insert-opening 3b is processed to be a circle, a quadrangle and so forth.

In addition, according to the aspect of the Embodiment 1, both the first guide 2b and the second guide 2a are installed, but only one of the first guide 2b and the second guide 2a can be installed.

The insert-opening 3b is in-place out of the optical axis (non-optical-axis) of the laser light to be connected. The position-difference h between the connection-opening 3a and the insert-opening 3b relative to the optical axis of the laser light is an optical axis gap h.

Next, referring to FIG. 1, the inventors set forth an action when the optical connector 1 outputs the laser light.

The optical fiber 4 is introduced into the insert-opening 3b along the first guide 2b and then, the tip of the optical fiber reaches to the connection-opening 3a along the second guide 2a. Then, the laser light propagates inside the optical fiber 4 and the laser output light is irradiated from the optical connector 1.

FIG. 2A is a cross-section view of the optical connector according to the aspect of the Embodiment 1 when the laser light is blocked. Referring to both FIGS. 2A, 2B, when blocking the laser light, the optical fiber 4 is pulled out from the connection-opening 3a through the second guide 2a, the insert-opening 3b and the first guide 2b. Then, the laser light that scatters or travels in the straight line inside the connection-opening 3a and passes therethrough contacts the proximity of the insert-opening 3b, which is different from the laser optical-axis, and scatters and results in diminishing or blocking of the laser light.

Accordingly, it is provided with the optical connector and the laser apparatus that facilitate the optical fiber 4 to be attached thereto and detached therefrom, are miniaturized with the simple structure, control the output of laser light and improve the safety therefor. In addition, following such a miniaturization, it is provided with the laser apparatus that is portable, workable even with a dry-cell battery and driven thereby.

In addition, with regard to a mounting method for the adapter 1b, the position is adjusted both the laser optical axis direction and the vertical direction, so that the optical connector 1 in which the laser light is diminished and blocked to have a desired light intensity is provided. For example, given the optical fiber 4 is inserted into the optical connector 1, the desired light intensity (over the class 1) is obtained outside through the optical fiber 4. In addition, given the optical fiber 4 is taken out from the optical connector 1, the light intensity meets the Class 1 laser specification.

The surface, on which the laser light of the adapter 1b shines, has an uneven (convex-and-concave) structure, so that the laser light scatters more and the diminishing and blocking (shielding) properties are improved. In addition, the surface area, which the laser light of the adapter 1b contacts, increases, is operative as a beam-dumper when the incident laser light has a high-power output.

In addition, according to the aspect of the Embodiment 1, the position between the connection-opening 3a and the insert-opening 3b is also set-up in accordance with the opening number NA (light receiving angle) of the optical fiber 4 to be used or the converging light NA (incident converging light angle) of the laser light. Accordingly, the adapter 1b more absolutely diminishes and blocks the laser light.

Specifically, if the optical axis gap, which is the position-difference between the connection-opening 3b and the optical axis of the laser light of the insert-opening 3b is h, the diameter of the connection-opening 3a is d, the distance between the insert-opening 3b and the connection-opening 3a is L, the laser incident converging angle or the light receiving angle is θ; and

H=L·tan θ+d (1)

is defined, and the insert-opening 3b is formed relative to the connection-opening 3a at the position defined using the above expression.

For example, if NA=0.2, d=0.3 mm and L=2 mm are given, h=0.71 mm is defined. If NA=0.1, d=0.3 mm and L=2 mm are given, h=0.5 mm is defined.

Further, referring to FIG. 3, when the optical axis gap h is set up over the defined value of the above h, the diminishing and blocking property of the laser light 5 is further improved. In such a case, the optical axis gap h that is a position-difference between the laser connection element 6 and the insert-opening 7 relative to the optical axis of the laser light is specified by the maximum value (allowable value, h=2R) of the bending R of the optical fiber 4 to be inserted.

### Embodiment 2

FIG. 4 is a view illustrating fiber-wiring inside the optical connector according to the aspect of the Embodiment 2. With regard to the optical connector 1 according to the aspect of the Embodiment 1, the optical fiber is taken out and put in along the optical axis direction of optical fiber, and instead, with regard to the optical connector according to the aspect of the Embodiment 2, the optical fiber is taken out and put in along the different direction from the optical axis direction of the laser light.

The optical connector according to the aspect of the Embodiment 2 comprises an optical adapter 1c and an optical adapter 1d. The optical adapter 1c and the optical adapter 1d are made of easily processable metal material such as copper, aluminum and so forth.

The optical adapter 1d comprises an insert-opening 3d into which the optical fiber 4 is inserted and a first guide 2d having a conical shape to guide the optical fiber 4 to the insert-opening 3d. An opening-angle of the conical-shape first guide 2d is processed to be e.g., 60-degrees and the hole (opening) diameter of the insert-opening 3d is slightly larger than the diameter of the optical fiber.

The optical adapter 1c comprises a connection-opening 3c that achieves positioning the tip of the optical fiber 4 inserted into the insert-opening 3d to the desired laser light connection position and a second guide 2c to guide the optical fiber 4, which is inserted into the insert-opening 3d, to the connection-opening 3b. The second guide 2c is in an approximately trapezoidal shape to introduce the optical fiber 4 from the insert-opening 3d to the connection-opening 3c.

The connection-opening 3c and the insert-opening 3d are in-place approximately orthogonal to each other and allows the optical fiber to be taken out and put in along the different direction from the optical axis of the laser light. In such a case, the insert-opening 3d is in-place on the non-optical-axis of the laser light to be connected. In addition, the positional relationship between the connection-opening 3c and the insert-opening 3d is determined based on the allowable range of the bending R of the optical fiber.

The optical connector according to the aspect of the Embodiment 2 is also operative to act as the same as the action of the optical connector according to the aspect of the Embodiment 1, so that the same effect is obtained.

In addition, referring to FIG. 4, the form of the optical fiber in-place inside the optical connector is mainly illustrated, but the optical connector of present invention is not limited to the form of the adapter illustrated in FIG. 4. In addition, the angle between the connection-opening 3c and the insert-opening 3d is not limited to 0-degrees.

In addition, the optical fiber 4 can be made of a glass-fiber, a plastic-fiber and so forth. When the optical fiber having a smaller bending R is used, the optical connector is further preferably miniaturized.

In addition, it is preferable that an adapter separated from the optical connector of the present invention to hold the optical fiber is used and the length of the optical fiber inserted into the optical connector is arbitrarily adjusted, i.e., as the tip of the optical fiber fits to the connection-opening or the beam converging position, so that the connection efficiency between the laser light and the optical fiber can be further increased.

### Embodiment 3

FIG. 5 is a view illustrating fiber-wiring inside the optical connector according to the aspect of the Embodiment 3. The optical connector, according to the aspect of the Embodiment 3, takes out and put in the optical fiber in the different direction from the optical axis direction of the laser light as well as the Embodiment 2.

The optical connector according to the aspect of the Embodiment 3 comprises an optical adapter 1e. The optical adapter 1e is made of easily processable metal material such as copper, aluminum and so forth.

The optical adapter 1e comprises an insert-opening 3f into which the optical fiber is inserted, a conical shape first guide 2f to guide the optical fiber 4 to the insert-opening 3f, a connection-opening 3e that achieves positioning the tip of the optical fiber 4 inserted into the insert-opening 3f to the desired laser light connection position and a second guide 2e to guide the optical fiber 4, which is inserted into the insert-opening 3f, to the connection-opening 3e.

The connection-opening 3e and the insert-opening 3f are in-place approximately in the 180-degrees opposite direction each other and allows the optical fiber to be taken out and put in along the different direction from the optical axis of the laser light. In such a case, the insert-opening 3f is in-place on the non-optical-axis of the laser light to be connected. In addition, the positional relationship between the connection-opening 3e and the insert-opening 3f is determined based on the allowable range of the bending R of the optical fiber.

The optical connector according to the aspect of the Embodiment 3 is also operative to act as the same as the action of the optical connector according to the aspect of the Embodiment 1, so that the same effect is attained.

In addition, the present invention is not limited to the above Embodiments 1 to 3. According to the aspect of the Embodiments 1 to 3, the second guide is installed, but e.g., the optical fiber is directly inserted into the insert-opening without installing the second guide.

(A Variety of Equipment Having the Optical Connector)

The laser device using the optical connector of the present invention provides the optical fiber with very high-freedom relative to the wavelength and light intensity from the laser light source or as the modified signal, so that such advantages is applied to an equipment (a guide light, an illumination device, an observation device for analysis and measurement, a process device, a medical therapy device, an optical communication device and so forth) in which the laser light propagates to the target.

For example, the laser apparatus using the optical connector is also be applied to an illumination device utilizing the light scattering technology based on an optical fiber and an illumination device having an excellent efficiency (of electricity and heat) as an LED alternative. In addition, with regard to the medical catheter lighting system, the connector and laser device of the present invention facilitates the catheter to be easily and safely attached and detached in the medical site.

Next, the inventors set forth a laser apparatus, an illumination apparatus, a processing apparatus, a therapeutic apparatus and an optical communication apparatus using the optical connector. Referring to FIG. 6, the laser apparatus comprises a laser light source 10, a convergence lens 11, an optical connector 1 and an optical fiber 4. The laser light source 10 emits the laser light to the optical connector 1 through the convergence lens 11. The optical fiber 4 is attachable to the optical connector 1 and detachable therefrom and the laser light from the laser light source 10 is incident thereinto.

The illumination apparatus comprises the laser light source 10, the convergence lens 11, the optical connector 1 and the optical fiber 4. A luminescent apparatus emits following irradiating the light including at least one of red, orange, yellow, green and blue light from the optical fiber 4.

The processing apparatus comprises the laser light source 10, the convergence lens 11, the optical connector 1, the optical fiber 4 and a laser processing unit. The processing unit processes the target object for processing using the laser light output from the optical fiber 4.

The therapeutic apparatus comprises the laser light source 10, the convergence lens 11, the optical connector 1, the optical fiber 4 and a laser therapeutic unit. The therapeutic unit treats the therapeutic target using the laser light output from the optical fiber 4.

The optical communication apparatus comprises the laser light source 10, the convergence lens 11, the optical connector 1 and the optical fiber 4 and an optical communication unit. The optical communication unit carries out optical communication using the laser light as a light signal.

### REFERENCE OF SIGNS

1 Optical connector
1a, 1b Optical adapter
2a Second guide
2b First guide
3a Connection-opening
3b Insert-opening
4 Optical fiber
5 Laser light
10 Laser Source
11 Convergence lens

It will be further understood by those of skill in the art that the apparatus and devices and the elements herein, without limitation, and including the sub components such as operational structures, circuits, communication pathways, and related elements, control elements of all kinds, display circuits and display systems and elements, any necessary driving elements, inputs, sensors, detectors, memory elements, processors and any combinations of these structures etc. as will be understood by those of skill in the art as also being identified as or capable of operating the systems and devices and subcomponents noted herein and structures that accomplish the functions without restrictive language or label requirements since those of skill in the art are well versed in related optical and laser connections, systems, and processing devices, computer and operational controls and technologies of such laser devices and all their sub components, including various circuits and combinations of circuits without departing from the scope and spirit of the present invention.

Although only a few embodiments have been disclosed in detail above, other embodiments are possible and the inventors intend these to be encompassed within this specification. The specification describes certain technological solutions to solve the technical problems that are described expressly and inherently in this application. This disclosure describes embodiments, and the claims are intended to cover any modification or alternative or generalization of these embodiments which might be predictable to a person having ordinary skill in the art.

Also, the inventors intend that only those claims which use the words “means for” are intended to be interpreted under 35 USC 112, sixth paragraph. Moreover, no limitations from the specification are intended to be read into any claims, unless those limitations are expressly included in the claims.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

1. An optical connector, comprising:
an insert-opening into which an optical fiber is inserted; and
a connection-opening that is open to introduce a tip of said optical fiber inserted into said insert-opening to a desired laser light connection position;
wherein, said insert-opening is in-place on a non-optical-axis of a laser light to be connected and a position-difference between said connection-opening and said insert-opening relative to an optical-axis of the laser light is larger than a predetermined value;
an optical axis of said optical fiber relative to said insert-opening and an optical axis of said optical fiber relative to said connection opening are parallel to a taking-out-and-putting-in direction of said optical fiber, said optical connector further comprising:
a first guide, having a conical shape, that is positioned to guide said optical fiber to said insert-opening; and
a second guide, having a conical shape, that is positioned to guide said optical fiber inserted into said insert-opening into said connection-opening.

2. (canceled)

3. The optical connector, according to claim 1, wherein:
when said position-difference between said connection-opening and said insert-opening relative to said optical-axis of said laser light is h, a diameter of said connection-opening is d, a distance between said insert-opening and said connection-opening is L; and
at least one angle, selected from a group of angles consisting of: a laser incident converging angle and a light receiving angle of said optical fiber is θ, wherein h≥L·tan θ+d is defined.

4. The optical connector, according to claim 1, wherein:
when said position-difference between said connection-opening and said insert-opening relative to said optical-axis of said laser light is h, h is larger than a bending R value of said optical fiber; and
h≥2R is defined.

5. A therapeutic apparatus, comprising:
said optical connector according to claim 1;
a laser light source that emits a laser light to said optical connector; and
an optical fiber that is attachable to said optical connector and detachable therefrom;
wherein said laser light from said laser light source is incident into said optical fiber.

6. A therapeutic apparatus, comprising:
said optical connector according to claim 1;
a laser light source that emits a laser light to said optical connector;
an optical fiber that is attachable to said optical connector and detachable therefrom;
a luminescent apparatus that emits following irradiation light of at least one selected from a group of following irradiation lights consisting of, a red light, an orange light, a yellow light, a green light, and a blue light from said optical fiber; and
wherein said laser light from said laser light source is incident into said optical fiber.

7. A processing apparatus, comprising:
said optical connector according to claim 1;
a laser light source that emits a laser light to said optical connector;
an optical fiber that is attachable to said optical connector and detachable therefrom;
a processing unit that processes a target object for processing using said laser light output from said optical fiber; and
wherein said laser light from said laser light source is incident into said optical fiber.

8. A therapeutic apparatus, comprising:
said optical connector according to claim 1;
a laser light source that emits a laser light to said optical connector;
an optical fiber that is attachable to said optical connector and detachable therefrom;
a therapeutic unit that processes a target object for therapy using said laser light output from said optical fiber; and
wherein said laser light from said laser light source is incident into said optical fiber.

9. A therapeutic apparatus comprising:
said optical connector according to claim 1;
a laser light source that emits a laser light to said optical connector;
an optical fiber that is attachable to said optical connector and detachable therefrom;
an optical communication unit that carries out optical communication using said laser light as a light signal; and
wherein said laser light from said laser light source is incident into said optical fiber.
